(19) United States
(12) Patent Application Publication
(10) Pub. No.: US 2012/0226136 A1
(43) Pub. Date: Sep. 6, 2012

(54) METHODS AND DEVICES OF DETECTION, GRADING, MONITORING, AND FOLLOW-UP OF FIBROSIS

(75) Inventors: Yixiang WANG, Hong Kong (CN); Jun YU, Hong Kong (CN); Jing YUAN, Hong Kong (CN)

(73) Assignee: THE CHINESE UNIVERSITY OF HONG KONG, HONG KONG (CN)

(21) Appl. No.: 13/411,800

(22) Filed: Mar. 5, 2012

Related U.S. Application Data
(60) Provisional application No. 61/449,479, filed on Mar. 4, 2011.

(51) Int. Cl. A61B 5/055

(52) U.S. Cl. 600/410

(57) ABSTRACT

Methods and devices of detection, grading, monitoring and follow-up fibrosis in a tissue or an organ of a subject using nuclear magnetic resonance imaging, particularly spin-lattice relaxation time in the rotating frame, which is also called spin lock relaxation time (T1rho).

### RELATED APPLICATIONS

This application claims priority under 35 U.S.C. §119(e) to U.S. Provisional Application No. 61/449,479, filed Mar. 4, 2011

### FIELD OF INVENTION

The present application relates to methods and devices of detection, grading, monitoring, and follow-up of fibrosis in a subject, particularly, methods and devices of detection, grading, monitoring, and follow-up of fibrosis in a subject using nuclear magnetic resonance imaging.

### BACKGROUND

Progressive fibrosis of liver, kidney, and other viscera often results in organ failure leading to death or the need for transplantation. These diseases affect millions worldwide. For example, liver fibrosis is the leading non-malignant gastrointestinal cause of death in the United States.

Liver fibrosis, a common feature of many chronic liver diseases, involves the accumulation of collagen, proteoglycans, and other macromolecules within the extracellular matrix. The accumulation of proteins in the extracellular matrix promotes the formation of scars that bridge together adjacent portal triads and central veins. Ultimately, progressive hepatic fibrosis leads to cirrhosis, a characteristic of most end-stage liver disease (1). Patients remain asymptomatic or have only mild, nonspecific symptoms until the development of cirrhosis. Cirrhotic patients may present with various sequelae of hepatic decompensation, including variceal hemorrhage, ascites, hepatic encephalopathy, and hepatic and renal failure. Cirrhosis is also a risk factor for the development of hepatocellular carcinoma. Originally considered to be irreversible, hepatic fibrosis is now regarded as a dynamic process with potential for regression (1).

To date, noninvasive diagnostic tests available from clinical practice are not sensitive or specific enough to detect occult liver injury at early or intermediate stages. Liver biopsy is the standard of reference for diagnosis and staging of liver fibrosis. However, it is an invasive procedure with possible side complications (2). Histologic assessment of fibrosis is also an inherently subjective process, and subjects to sampling variability (3). The extent of variations from observer interpretation by expert histopathologists may be as high as 20% (4). To better manage individuals with progressive fibrosis, especially those benefit from early intervention, a reproducible and reliable noninvasive method is needed to evaluate disease progression, to monitor responses to drug treatment.

### SUMMARY

Accordingly, disclosed herein are methods and devices of detection, grading, monitoring and follow-up of fibrosis in a subject using nuclear magnetic resonance imaging.

According to one aspect disclosed herein, there is provided a method of detecting or monitoring fibrosis in a tissue or an organ of a subject using nuclear magnetic resonance imaging, comprising generating a spin-lattice relaxation time in the rotating frame (T1rho) weighted image of the region of interest (ROI) in said tissue or organ of the subject, measuring the T1rho value of the region of interest (ROI) in said tissue or organ of the subject via an algorithm, and comparing the T1rho value in the tissue or organ of the subject with a normal standard of the T1rho value in said tissue or organ, or with another T1rho value obtained from the tissue or organ of the subject at other time points, wherein an increase of the T1rho value in the tissue or organ of the subject compared with the normal standard indicates that the subject is suffering from the tissue or organ fibrosis; or an increase or decrease of the T1rho value in the tissue or organ of the subject compared with the T1rho value obtained at other time points indicates the progression or regression of the tissue or organ fibrosis.

According to another aspect disclosed herein, there is provided a device for detecting or monitoring fibrosis in a tissue or an organ of a subject using nuclear magnetic resonance imaging, comprising:

a means for generating a spin-lattice relaxation time in the rotating frame (T1rho) weighted image of the region of interest (ROI) in said tissue or organ of the subject;

a means for measuring the value of said T1rho of the region of interest (ROI) in said tissue or organ of the subject by running an algorithm; and

a means for comparing the T1rho value in the tissue or organ of the subject with a normal standard of T1rho value in said tissue or organ, or with another T1rho value obtained from the tissue or organ of the subject at other time points;

wherein an increase of the T1rho value in the tissue or organ of the subject compared with the normal standard indicates that the subject is suffering from the tissue or organ fibrosis; or an increase or decrease of the T1rho value in the tissue or organ of the subject compared with the T1rho value obtained at other time points indicates the progression or regression of the tissue or organ fibrosis.

In an embodiment, an extent of the increase of T1rho value from normal ranges indicates the severity of the fibrosis, i.e. the larger the increase, the more severe the fibrosis; and the rate of the increase or decrease of T1rho value over time indicates the rate of the progression or regression of the tissue or organ fibrosis.

In another embodiment, the algorithm is to generate a T1rho map of the region of interest (ROI) on a pixel-by-pixel basis from the acquired T1rho-weighted images using a T1rho relaxation theoretical model, and obtain the T1rho value of the region of interest (ROI) from the T1rho map; and.

In a further embodiment, the algorithm is to obtain the T1rho value from all the pixels of the region of interest (ROI) in the acquired T1rho images using a T1rho relaxation theoretical model

In another embodiment, the T1rho relaxation theoretical model is mono-exponential decay model.

In other embodiments, the T1rho relaxation theoretical model is multi-exponential decay model. In a preferred embodiment, the T1rho mono-exponential decay model is described by the following equation, wherein TSL is time of spin lock pulse:

M(TSL)=M0*exp(−TSL/T1rho).

In a preferred embodiment, a spin-lock pulse is used together with a 2D or 3D MRI pulse sequence. In a preferred embodiment, the spin-lock pulse is a rotary echo or other spin-lock pulses for generating a T1rho image. In a preferred embodiment, the 2D or 3D MRI pulse sequence is selected from the group consisting of spin echo (SE), fast spin echo (FSE), gradient echo (GRE), echo planar imaging (EPI) sequences and a 3D balanced fast field echo (bFFE) sequence.

In still another embodiment, a rotary echo spin-lock pulse is used together with a 3D balanced fast field echo (bFFE) sequence for generating a T1rho weighted image.

In other embodiment, the fibrosis is selected from the group consisting of liver, kidney, and other viscera fibrosis, preferably the fibrosis is liver fibrosis.

In an embodiment, the subject comprises a human or an animal.

In an embodiment, the normal standard of T1rho value is a T1rho value determined from a normal subject or a group of the normal subjects not having the fibrotic tissue or organ.

In an embodiment, the current and other time points for acquiring T1rho weighted images can have a time interval as desired in the detection, such as four weeks, two months, three months, four months, five months, six months, one year, two years, etc.

In an embodiment, the magnetic field strength used for acquiring a T1rho weighted image is selected from the group consisting of: low field below 1.5T, high field between 1.5T to 3T, and ultra high field over 3T.

### BRIEF DESCRIPTION OF DRAWINGS

FIG. 1 shows five regions-of-interest (ROIs) are placed on each slice of the rat liver parenchyma region for each imaging sequence to quantify liver signal intensity on T1 weighted image (a), and T1rho value on T1rho maps (b).

FIG. 2 shows longitudinal follow-up MRI measurement of liver absolute T1rho value (A) and normalized liver signal on conventional T1 weighted images (B). At day 8 post biliary duct ligation (BDL), though the BDL rat livers (n=8) tend to have higher liver T1rho value, half of them overlaps with the values of sham operated rat liver (n=4). At day 15, seven BDL rat livers had higher T1rho value than sham operated rat livers. BDL rat liver (n=6) T1rho values further increased on day 21. The sham operated rats were not examined on day 21. There was a clear separation of the two groups of rats on day 29. Normalized liver signal on conventional T1 weighted images does not show BDL rats and sham operated rats separation (B). Solid line: BDL rats; Dotted line: sham operated rats.

FIG. 3 shows livers absolute T1rho value (A) and normalized liver signal intensity on conventional T1 weighted images (B) on day 24 and day 38 post biliary duct ligation (BDL) surgery. Liver T1rho value separated the BDL rats (n=5) and sham-operated control rats (n=5) clearly (A). Liver T1rho values increase from day 24 to day 38 for all BDL rats, while those of control rats remain consistent. Normalized liver signal on conventional Ti weighted images does not show BDL rats and sham-operated rats separation (B). Solid line: BDL rats; Dotted line: sham operated rats.

FIG. 4 shows shaded T1rho maps of a sham-operated rat liver (control, upper row) and a biliary duct ligation (BDL) rat liver (lower row) 24 days post surgery. BDL rat liver demonstrates higher T1rho value (dark shade) than the control rat liver (light shade). Arrow: dilated biliary duct. Dotted arrow: gas in the stomach.

FIG. 5 shows H&E stained sections (original magnification ×100). Sham-operated rats show normal liver histology (A to D). Bile duct proliferation and hepatic inflammatory infiltration are observed in bile duct ligation (BDL) rats at day 8, 15, 24, and 38, respectively; and there is an apparent progression in the severity of liver pathology from day 8 to day 24 (E to H).

FIG. 6 shows collagen deposition with Picrosirius red stain of bile duct ligation (BDL) rats and sham-operated rats (control) at different time points (original magnification ×100). Normal liver tissue with Picrosirius red-positive fibrils around central vein and the portal triads (A to D) are observed in sham-operated rats, while minimal collagen fibers are observed in BDL rats at day 8 (E), obvious liver fibrosis is found at day 15, 24, and day 38 (F to H). There is an apparent progression of liver fibrosis from day 8 to day 24 in BDL rats.

FIG. 7 shows liver collagen area at different time points post surgery. BDL, bile duct ligation; Data are mean±SD.

### DETAILED DESCRIPTION OF THE EMBODIMENTS TERMS

As used herein, the term “relaxation” describes several processes by which nuclear magnetization prepared in a non-equilibrium state return to the equilibrium distribution. In other words, relaxation describes how fast spins “forget” the direction in which they are oriented. The rates of this spin relaxation can be measured in imaging applications.

Different physical processes are responsible for the relaxation of the components of the nuclear spin magnetization vector M parallel and perpendicular to the external magnetic field, B₀ (which is conventionally oriented along the z axis). These two principal relaxation processes are termed T₁ and T₂ relaxation respectively.

As used herein, a longitudinal (or spin-lattice) relaxation time “T₁”is the decay constant for the recovery of the z component of the nuclear spin magnetization, M_{z}, towards its thermal equilibrium value, M_{z,eq}. In general,

M_{z}(t)=M_{z,eq}−[M_{z,eq}−M_{z}(0)]e^{-t/T}^{₁}

In specific cases:
If M has been tilted into the xy plane, then M_{z}(0)=0 and the recovery is simply

M_{z}(t)=M_{z,eq}(1−e^{-t/T}^{₁})

i.e. the magnetization recovers to 63% of its equilibrium value after one time constant T₁.
In the inversion recovery experiment, commonly used to measure T₁ values, the initial magnetization is inverted, M_{z}(0)=−M_{z,eq}, and so the recovery follows

M_{z}(t)=M_{z,eq}(1−2e^{-t/T}^{₁})

T₁ relaxation involves redistributing the populations of the nuclear spin states in order to reach the thermal equilibrium distribution. By definition this is not energy conserving. Moreover, spontaneous emission is negligibly slow at NMR frequencies. Hence truly isolated nuclear spins would show negligible rates of T₁ relaxation. However, a variety of relaxation mechanisms allow nuclear spins to exchange energy with their surroundings, the lattice, allowing the spin populations to equilibrate. The fact that T₁ relaxation involves an interaction with the surroundings is the origin of the alternative description, spin-lattice relaxation.

Note that the rates of T₁ relaxation are generally strongly dependent on the NMR frequency and so vary considerably with magnetic field strength B. Small amounts of paramagnetic substances in a sample speed up relaxation very much. By degassing, and thereby removing dissolved Oxygen, the T₁/T₂ of liquid samples easily goes up to an order of ten seconds.

As used herein the term “transverse or spin-spin relaxation time T₂” is the decay constant for the component of M perpendicular to B₀, designated M_{xy}, or M_{T}. For instance, initial xy magnetization at time zero will decay to zero (i.e. equilibrium) as follows:

M_{xy}(t)=M_{xy}(0)e^{-t/T}^{₂}

i.e. the transverse magnetization vector drops to 37% of its original magnitude after one time constant T₂.

T₂ relaxation is a complex phenomenon, but at its most fundamental level, it corresponds to a decoherence of the transverse nuclear spin magnetization. Random fluctuations of the local magnetic field lead to random variations in the instantaneous NMR precession frequency of different spins. As a result, the initial phase coherence of the nuclear spins is lost, until eventually the phases are disordered and there is no net xy magnetization. Because T₂ relaxation involves only the phases of other nuclear spins it is often called “spin-spin” relaxation.

T₂ values are generally much less dependent on field strength, B, than T₁ values. Relaxation in the rotating frame, T_{1ρ}

The discussion above describes relaxation of nuclear magnetization in the presence of a constant magnetic field B₀. This is called relaxation in the laboratory frame. Another technique, called relaxation in the rotating frame, is the relaxation of nuclear magnetization in the presence of the field B₀ together with a time-dependent radiofrequency (RF) pulse magnetic field B₁. The field B₁ rotates in the plane perpendicular to B₀ at the Larmor frequency of the nuclei in the B₀, and the nuclei also rotate in the plane perpendicular to B₀ in the same rate as field B₁ so that the nucleic keep relatively still with B₁. The magnitude of B₁ is typically much smaller than the magnitude of B₀. Under these circumstances the relaxation of the magnetization is similar to laboratory frame relaxation in a field B₁. The decay constant for the recovery of the magnetization component along B₁ is called the spin-lattice relaxation time in the rotating frame or the spin lock relaxation time and is denoted T_{1ρ} (T1rho). Relaxation in the rotating frame is useful because it provides information on slow motions of nuclei. In biological tissues, T1rho contrast is sensitive to macromolecular composition and provides contrast different from conventional T1 or T2-based imaging methods.

The term “subject” used herein refers to a human or an animal. The animal is preferably selected from the group consisting of a monkey, dog, horse, deer, pig, guinea pig, sheep, goat, cattle, buffalo, rabbit, cat, rat and mouse.

### Detailed Descriptions of Embodiments

Disclosed here are methods and devices of detection, grading, monitoring and follow-up of fibrosis in a subject using nuclear magnetic resonance imaging.

According to one aspect disclosed herein, there is provided a method of detecting or monitoring fibrosis in a tissue or an organ of a subject using nuclear magnetic resonance imaging, comprising generating a spin-lattice relaxation time in the rotating frame (T1rho) weighted image of the region of interest (ROI) in said tissue or organ of the subject, measuring the T1rho value of the region of interest (ROI) in said tissue or organ of the subject via an algorithm, and comparing the T1rho value in the tissue or organ of the subject with a normal standard of the T1rho value in said tissue or organ, or with another T1rho value obtained from the tissue or organ of the subject at other time points, wherein an increase of the T1rho value in the tissue or organ of the subject compared with the normal standard indicates that the subject is suffering from the tissue or organ fibrosis; or an increase or decrease of the T1rho value in the tissue or organ of the subject compared with the T1rho value obtained at other time points indicates the progression or regression of the tissue or organ fibrosis.

According to another aspect disclosed herein, there is provided a device for detecting or monitoring fibrosis in a tissue or an organ of a subject using nuclear magnetic resonance pin-lattice relaxation time in the rotating frame (T1rho) weighted imaging, comprising:

a means for generating a spin-lattice relaxation time in the rotating frame (T1rho) weighted image of the region of interest (ROI) in said tissue or organ of the subject;

a means for measuring the value of said T1rho of the region of interest (ROI) in said tissue or organ of the subject by running an algorithm; and

a means for comparing the T1rho value in the tissue or organ of the subject with a normal standard of T1rho value in said tissue or organ, or with another T1rho value obtained from the tissue or organ of the subject at other time points;

wherein an increase of the T1rho value in the tissue or organ of the subject compared with the normal standard indicates that the subject is suffering from the tissue or organ fibrosis; or an increase or decrease of the T1rho value in the tissue or organ of the subject compared with the T1rho value obtained at other time points indicates the progression or regression of the tissue or organ fibrosis.

In an embodiment, the T1rho value is reflected by signal intensity on T1rho weighted magnetic resonance images.

In an embodiment, the fibrosis is selected from the group consisting of liver, kidney, and other viscera fibrosis, preferably liver fibrosis.

In one embodiment, the normal standard of T1rho is a T1rho value determined from a normal subject or a group of the normal subjects not having the fibrotic tissue or organ and can be determined by various methods including those for measuring T1rho as disclosed herein.

In one embodiment of the invention, the current and other time points for generating T1rho weighted images can have a time interval as desired in the detections, such as four weeks, two months, three months, four months, five months, six months, one year, two years, etc.

In an embodiment, MRI data acquisition can be performed at any main magnetic field strength, such as low field belowl.5T, high field between 1.5T to 3T, and ultra high field over 3T with a suitable spin-lock frequency to the specific magnetic field.

In one embodiment, a spin-lock pulse such as a rotary echo spin-lock pulse, can be implemented in various MM 2D or 3D pulse sequences, such as spin echo (SE), fast spin echo (FSE), gradient echo (GRE), echo planar imaging (EPI) sequences and a 3D balanced fast field echo (bFFE) sequence, for T1rho imaging. Preferably, a rotary echo spin-lock pulse is implemented in a 3-D balanced fast field echo (bFFE) sequence for T1rho imaging. In other embodiments, spin-lock frequency can be set in the range of from tens of Hertz to several Kilohertz under the constraint of the allowable specific-absorption-rate (SAR) according to the appropriate MRI safety regulations. For example, Spin-lock frequency can be set as over 2000 Hz at 0.5T, or 1000 Hz at 1.5T. In a specific embodiment, for the liver scan, the spin-lock frequency can be set as 500 Hz at 3T. Times of spin-lock pulse (TSL) can be set for example as 0 ms, 10 ms, 20 ms, 30 ms, 40 ms, and 50 ms for T1rho mapping. More or less TSLs can be used according to the specific application.

T1rho imaging can be performed generally using various MRI 2D/3D pulse sequences. In a specific embodiment of the b-FFE preparation module, a half-alpha pulse and startup pulses with dummy-scans are used to approach the steady-state but with T1ρ-weighted preparation maintained. The subsequent normal phase alternating bFFE readout is used for acquisition. TI (delay time) after acquisition is set as long as to restore equilibrium magnetization prior to the next T1ρ preparation, for example 6000 ms. Short TE (echo time) and TR (repetition time) are set according to the specific scan requirement, for example, TE and TR are set as 2.6 ms and 5.2 ms respectively for liver scan using the 3T scanner. A voxel size is determined by the requirement on the spatial resolution, signal-to-noise ratio (SNR) and scan time, such as 0.50×0.62×2.00 mm³ for liver. Similarly, the flip angle can be set as 40 degree and the number of signal average (NSA) can be set as 4, also dependent on the specific scan requirement. Images are processed using a software program on the MRI console or off-line. For example, a home-made IDL program (ITTVIS, Boulder, Colo. USA) or Matlab (MathWorks, Natick MA USA) can be used to process the images of liver to generate T1rho maps.

In still another embodiment, T1rho maps of pixel-by-pixel are generated by fitting the imaging signal intensity of each pixel obtained at different times of spin lock (TSL) using a mono-exponential decay model as described by the following equation:

M(TSL)=M0*exp(−TSL/T1rho).

The equation is linearized by logarithm and then used to generate T1rho maps by fitting all pixel intensity data as a function of TSL time using linear regression. T1rho is calculated as −1/slope of the straight-line fit.

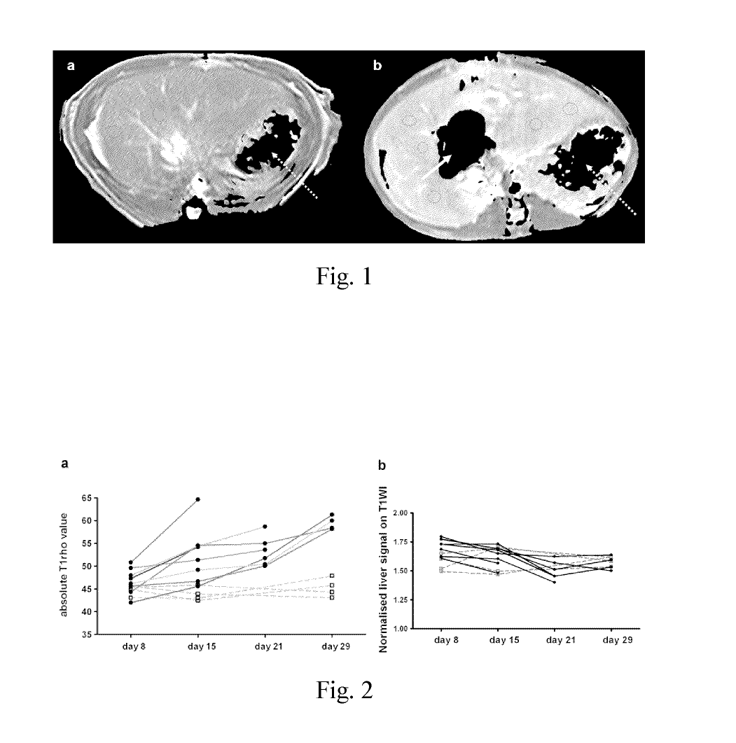

To quantify tissue signal intensity on T1rho value on T1rho maps, as an example, five regions-of-interest (ROIs) are placed on each slice of the tissue parenchyma region (FIG. 1), leading to a total of 25 ROIs from each liver for each imaging sequence if five slices are taken. For T1rho images, the mean value of these 25 ROIs is regarded as the T1rho value of the subject.

### EXAMPLES

### Methods

It is known that biliary duct ligation (BDL) in rats results in cholestatic liver injury, fibrosis and cirrhosis (6). This study explored the role of MR T1rho imaging in liver fibrosis evaluation using the rat BDL liver fibrosis model.

The protocols and procedures were approved by the local Animal Experimentation Ethics Committee. Seventy male Sprague-Dawley rats with weight of 200-250 g were used. The animals were housed on a 12-hour light/12-hour dark cycle in an air-conditioned room at 25° C. Food and water were available ad libitum. Bile duct ligation (BDL) was performed under general anesthesia. An upper abdominal incision was achieved using a sterile technique, and the common bile duct was isolated and double-ligated close to the liver. The bile duct was severed between the two ligatures. Sham operation was performed in an identical manner, with the exception of ligation and transection of the bile duct.

### MRI Studies

Two animal studies were performed with an interval of one month apart. The first study involved longitudinal MRI follow-up of BDL rats and sham operated control rats on day 8, day 15, day 21, and day 29 post surgery. The second study involved MRI study of another batch of BDL rats and sham operated rats on day 24 and day 38 post surgery. MRI data acquisition was performed on a 3T clinical scanner (Achieva, Philips Healthcare, Best, The Netherlands). After anesthesia, animals were positioned supine and a human wrist radiofrequency (RF) coil was used as the signal transmitter and receiver. Five axial slices were selected to cut through liver. T1rho mapping and T1 weighted images were obtained.

T1rho Measurement

For T1rho measurement, a rotary echo spin-lock pulse was implemented in a 3D balanced fast field echo (bFFE) sequence. Spin-lock frequency was set as 500 Hz and the spin-lock times of 1 ms, 10 ms, 20 ms, 30 ms, 40 ms, and 50 ms were used for T1rho mapping. In the b-FFE preparation module, a half-alpha pulse and startup pulses with dummy-scans were used to approach the steady-state but with T1rho -weighted preparation maintained. The subsequent normal phase alternating bFFE readout was used for acquisition. TI (delay time) after acquisition was set as 6000 ms to restore equilibrium magnetization prior to the next T1rho preparation. TE (echo time) and TR (repetition time) were 2.6 ms and 5.2 ms respectively. The voxel size was 0.50×0.62×2.00 mm³. The flip angle was 40 degree and the number of signal average (NSA) was 4. All images were processed using a home-made IDL program (ITTVIS, Boulder, Colo. USA) to generate T1rho maps. T1rho maps of pixel-by-pixel are generated by fitting the imaging signal intensity of each pixel obtained at different times of spin lock (TSL) using a mono-exponential decay model as described by the following equation:

M(TSL)=M0*exp(−TSL/T1rho).

The equation was linearized by logarithm and then used to generate T1rho maps by fitting all pixel intensity data as a function of TSL time using linear regression. T1rho was calculated as −1/slope of the straight-line fit.

Conventional TI Measurement

Conventional Ti weighted images of rat livers were acquired by turbo spin echo (TSE) sequences with TSE factor=3, TE/TR=10 ms/400 ms, voxel size=0.50×0.64×3 mm³, and NSA=2.

Morphology

The morphology of the liver on Ti weighted MRI images, regions-of-interest (ROI) measurement of absolute liver T1rho, and normalized liver signal on conventional T1 weighted images were assessed by a radiologist with experience in small animal imaging. To quantify liver signal intensity on Ti weighted image, and T1rho value on T1rho maps, five regions-of-interest (ROI) were placed on each slice of the liver parenchyma region (FIG. 1), leading to a total of 25 ROIs from 5 slices. For T1rho, the mean value of these 25 ROIs was regarded as the value of the rat. For T1 weighted images, the mean signal intensity of these ROIs was normalized by the signal intensity of the back muscle on the same image, i.e. the ratio of liver signal intensity and back muscle intensity was obtained.

Histological Analysis

For histology analysis, the animals were sacrificed on day 8, 15, 24, and 38 post surgery, respectively (n=6 per time point for BDL and control rats). Liver specimens were fixed in 4% phosphate-buffered formaldehyde and embedded in paraffin. Sections of 5 μm thick were dewaxed in xylene and rehydrated in a series of ethanol. In addition to standard Haematoxylin- and Eosin-(H&E) staining, sections were also processed with 0.1% Picrosirius red for collagen visualization. Histomorphometric analysis was carried out on a computerized image analysis system comprised of a photomicroscope and digital camera (Axiocam, Carl Zeiss Microscopy, Oberkochen, Germany) and Bioquant Nova Prime software (Bioquant Image Analysis Corporation, TN). Briefly, the entire liver section on a slide was captured by consecutive fields, each at a magnification of ×100, with no overlapping. The mean of red color stained area of all fields in each section was calculated. The mean area of fibrosis in μm² per field was calculated for each liver section.

### Statistics

Repeated measures of Analysis of Variance (ANOVA) were used. Treatment groups were compared at each time point and tests for linear trend were adopted across time in each group. All statistical analyses were performed using the statistical package SAS, version 9.1 (SAS Institute, Inc., Cary, N.C.). An α level of 5% was used as the level of significance.

### Results

### MRI

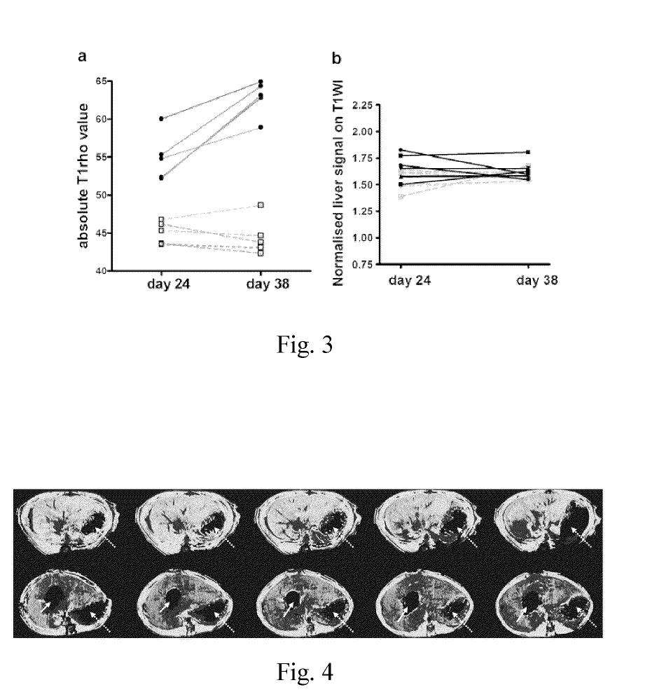

In the first study, on day 8 post surgery, though the BDL rats tended to have higher liver T1rho value, these two groups cannot be separated (46.7±2.9 vs 44.7±1.2 ms, P>0.05, FIG. 2A, Table 1). On day 15, one BDL rat with a lowest liver T1rho value on day 8 had liver T1rho value overlapped with those of control rats, while the rest BDL rat livers had higher T1rho values as compared to those of control rats (52.6±6.0 vs 43.8±1.5 ms, P=0.001). There was a clear separation between these two groups of rats on day 29 (59.5±1.5 vs 45.0±1.7 ms, P<0.001, FIG. 2A, Table 1). In the second study, T1rho values clearly separated BDL rat livers and control rat livers (FIG. 3A, Table 2). Liver T1rho values increased from day 24 (55.7±3.6 ms) to day 38 (62.3±2.2 ms, P=0.015) for all BDL rats, while those of the control rats remained unchanged (45.1±1.5 vs 44.4±2.3 ms, P=0.3, Table 2).

The resultant conventional T1 weighted images from rat liver were expressed as normalized liver signal (i.e. signal intensity ratio of liver/muscle). There was no difference in normalized liver signal between BDL rats and sham operated rats (FIG. 2B, FIG. 3B, P>0.05).

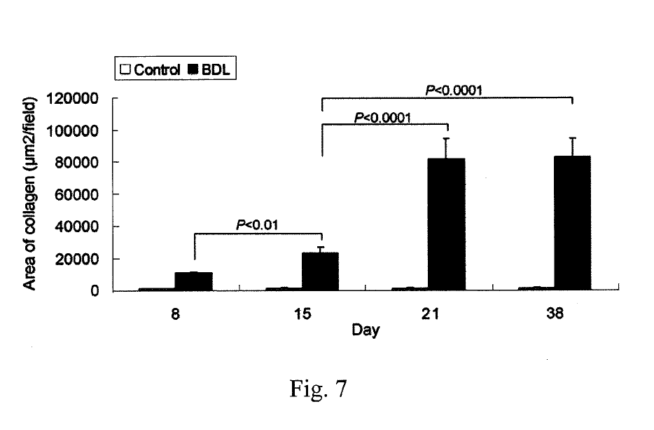

With morphological assessment all BDL rats did not show apparent signs of liver cirrhosis, except the biliary duct dilatation caused by the surgery (FIG. 7, FIG. 1).

While there existed a T1rho difference, color coded T1rho maps is able to differentiate BDL rat liver and control liver (FIG. 4).

TABLE 1
Absolute rat liver T1rho values at day 8, day 15, day 21, and
day 29 post biliary duct ligation surgery or sham surgery.

| | day 8 | day 15 | | day 29 |
|---|---|---|---|---|
| Sham operated rats | (n = 4) | (n = 4) | | (n = 4) |
| Mean ± SD (millisec) | 44.7 ± 1.2 | 43.8 ± 1.5 | N/A | 45.0 ± 1.7 |
| Range (millisec) | 43~45.6 | 43~45.8 | N/A | 43.1~46.8 |
| | day 8 | day 15 | day 21 | day 29 |
| Biliary duct ligation rats | (n = 8) | (n = 8) | (n = 6) | (n = 4) |
| Mean ± SD (millisec) | 46.7 ± 2.9 | 52.6 ± 6.0 | 53.2 ± 3.3 | 59.5 ± 1.5 |
| Range (millisec) | 42~50.8 | 45.6~64.6 | 50~58.7 | 58.1~60 |

The rat liver T1rho value at day 8 between the sham-operated control and biliary duct ligation (BDL) groups is not significantly different (P=0.40). This difference becomes significant at day 15 (P=0.001) and day 29 (P<0.001). The control rats were not MR imaging examined on day 21. There is no significant difference at three time points for the control rats (P=0.83). The trend for BDL rat liver T1rho value increasing at the four time points is significant (P<0.001). Repeated measures of Analysis of Variance (ANOVA) were used. Treatment groups were compared at each time point and tests for linear trend were adopted across time in each group. All statistical analyses were performed using the statistical package SAS, version 9.1 (SAS Institute, Inc., Cary, N.C.). An a level of 5% was used as the level of significance (same for Table 2).

TABLE 2
Absolute rat liver T1rho values at day 24 and day 38
post biliary duct ligation surgery or sham surgery.

| | day 24 | day 38 |
|---|---|---|
| | (n = 5) | (n = 5) |
| Sham operated rats | | |
| Mean ± SD(millisec) | 45.1 ± 1.5 | 44.4 ± 2.3 |
| Range (millisec) | 43.6~46.8 | 42.3~48.2 |
| Biliary duct ligation rats | | |
| Mean ± SD (millisec) | 55.7 ± 3.6 | 62.3 ± 2.2 |
| Range (millisec) | 52.8~61.1 | 58.6~64.0 |

The rat liver T1rho value at day 24 and day 38 between the sham operated and biliary duct ligation (BDL) groups is significantly different (P=0.0001 and P<0.0001 respectively). There is no significant difference at the two time points for the control rats (P=0.30). The trend for BDL rat liver T1rho value increasing at the two time points is significant (P=0.015).

### Histology and Histomorphometry

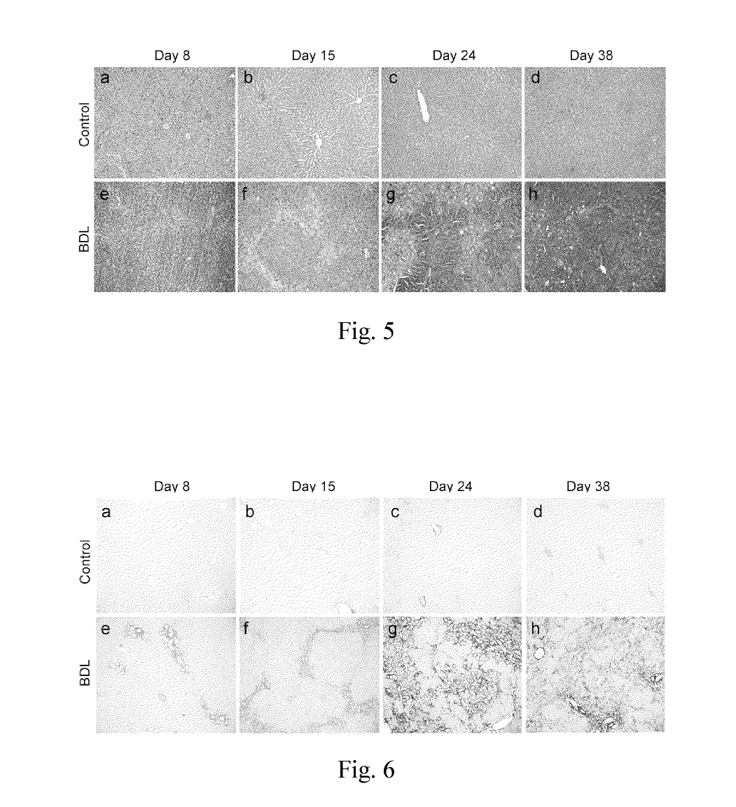

H&E staining revealed hepatic histology was within normal limits in sham-operated control rats at different time points (FIG. 5A-D). While, at day 8 following BDL, proliferation of bile duct and inflammatory cell infiltration around the portal triads were observed in operated rats (FIG. 5E); all rats at day 15 after BDL developed liver fibrosis with development of fibrous septa, loss of hepatocytes and pseudo-lobule formation with a background of inflammatory infiltration (FIG. 5F); all rats at day 21 and 38 following BDL had biliary cirrhosis and cirrhosis nodular with intense ductular proliferation, fibrotic bridges and more pronounced inflammatory infiltration (FIGS. 5G and H).

With Picrosirius red staining, no abnormality in parenchymal architecture was observed among the liver of sham-operated rats (FIG. 6A-D). Eight days after BDL, collagen fibers could be seen in BDL rat livers (FIG. 6E). Rats developed liver fibrosis with thick, and often complete fibrotic septa at day 15 following BDL (FIG. 6F). This was associated with a significant increase in collagen (FIG. 7) as compared with rats treated with BDL for 8 days (P<0.01). Rats subjected to BDL for 24 or 38 days had more marked liver fibrosis/cirrhosis (FIGS. 6G and H) consistent with significantly elevated collagen areas (P<0.0001) compared with rats treated with BDL for 15 days (FIG. 7). There was no significant difference for collagen areas between day 24 and day 38 (P>0.05) (FIG. 7).

### Discussion

Results from the current study showed rat liver tissue demonstrates proliferation of bile duct and inflammatory cell infiltration around the portal triads on day 8 post BDL surgery, however, liver fibrosis was minimal. At this time-point, the BDL rats demonstrated a trend to have higher liver T1rho values than the control rats (FIG. 2A). On day 15, BDL rats had liver fibrosis with the development of fibrous septa, loss of hepatocytes, and a background of inflammatory infiltration. BDL rats can be largely separated from normal control rats based on liver MR T1rho imaging at this time point. The degree of liver fibrosis post BDL surgery may show inter-animal variance, particularly in the early stage. It is interesting to note that histomorphometric analysis of collagen area on Picrosirius red histology showed that there was no increase of collagen content between day 21 and day 38 (FIG. 7). In contrast, MRI showed an increase of liver T1rho value during day 24 and day 38 suggesting a liver fibrosis progression (FIG. 3). These results suggest that liver fibrosis is associated with T1rho value increase, and MR T1rho imaging can be sensitive in evaluation of liver fibrosis progression. MR T1rho imaging is a non-invasive technique. It does not involve intravenous injection of contrast agents or external driver such as in the case of elastography.

From the above description, it will be apparent to those skilled in the relevant arts that the present methods have an advantage and benefits over prior art methods for characterizing the fibrosis. Specifically, the present methods provide a non-invasion measurement of fibrosis. Now that embodiments of the inventions have been described, it will be apparent to those skilled in the relevant arts that numerous modifications and variations may be made without departing from the basic inventive concepts. While the embodiments are described in relation to the liver, it is envisaged that the methods may be used for other parts of the body or structures that undergo fibrosis. All such modifications and variations that would be obvious to a person of ordinary skill in the art are deemed to be within the scope of the present invention the nature of which is to be determined from the above description and the appended claims.

### References:

1. Wallace K, Burt A D, Wright M C. Liver fibrosis. Biochem Genet 2008; 411:1-18

2. Janes C H, Lindor K D. Outcome of patients hospitalized for complications after outpatient liver biopsy. Ann Intern Med 1993; 118:96-98

3. Poniachik J, Bernstein D E, Reddy K R, Jeffers L J, Coelho-Little M E, Civantos F, et al. The role of laparoscopy in the diagnosis of cirrhosis. Gastrointest Endosc 1996; 43:568-571

4. Bravo A A, Sheth S G, Chopra S. Liver biopsy. N Engl J Med 2001; 344: 495-500

5. Duvvuri U, Poptani H, Feldman M, Nadal-Desbarats L, Gee M S, Lee W M, et al. Quantitative T1rho magnetic resonance imaging of RIF-1 tumors in vivo: detection of early response to cyclophosphamide therapy. Cancer Res. 2001; 61:7747-53

6. Yu J, Hui A Y, Chu E S, Go M Y, Cheung K F, Wu C W, et al. The anti-inflammatory effect of celecoxib does not prevent liver fibrosis in bile duct-ligated rats. Liver Int. 2009; 29:25-36

7. Talwalkar J A, Yin M, Fidler J L, Sanderson S O, Kamath P S, Ehman R L. Magnetic resonance imaging of hepatic fibrosis: emerging clinical applications. Hepatology. 2008; 47:332-42

8. Ito K, Mitchell D G. Imaging diagnosis of cirrhosis and chronic hepatitis. Intervirology 2004; 47: 134-143.

9. Salameh N, Larrat B, Abarca-Quinones J, Pallu S, Dorvillius M, Leclercq I, et al. Early detection of steatohepatitis in fatty rat liver by using MR elastography. Radiology 2009; 253:90-97

10. Bonekamp S, Kamel I, Solga S, Clark J. Can imaging modalities diagnose and stage hepatic fibrosis and cirrhosis accurately? J Hepatol. 2009; 50:17-35

What is claimed is:

1. A method of detecting or monitoring fibrosis in a tissue or an organ of a subject using nuclear magnetic resonance imaging, comprising generating a spin-lattice relaxation time in the rotating frame (T1rho) weighted image of the region of interest (ROI) in said tissue or organ of the subject, measuring the T1rho value of the ROI in said tissue or organ of the subject via an algorithm, and comparing the T1rho value in the tissue or organ of the subject with a normal standard of T1rho value in said tissue or organ, or with another T1rho value obtained from the tissue or organ of the subject at other time points, wherein an increase of the T1rho value in the tissue or organ of the subject compared with the normal standard indicates that the subject is suffering from the tissue or organ fibrosis; or an increase or decrease of the T1rho value in the tissue or organ of the subject compared with the T1rho value obtained at other time points indicates the progression or regression of the tissue or organ fibrosis.

2. The method according to claim 1, wherein an extent of the increase of T1rho value from normal ranges indicates the severity of the fibrosis, and the rate of the increase or decrease of T1rho value over time indicates the rate of the progression or regression of the tissue or organ fibrosis.

3. The method according to claim 1, wherein the T1rho value is reflected by signal intensity on the T1rho weighted image.

4. The method according to claim 1, wherein the algorithm is selected from the group consisting of:
generating a T1rho map of the region of interest (ROI) on a pixel-by-pixel basis from the acquired T1rho-weighted images using a T1rho relaxation theoretical model, and obtaining the T1rho value of the region of interest (ROI) from the T1rho map; and
obtaining the T1rho value from all the pixels of the region of interest (ROI) in the acquired T1rho images using a T1rho relaxation theoretical model.

5. The method according to claim 4, wherein the T1rho relaxation theoretical model is a mono-exponential decay model or a multi-exponential decay model.

6. The method according to claim 5, wherein the T1rho mono-exponential decay model is described by the following equation, wherein TSL is time of spin lock pulse:
M(TSL)=M0*exp(−TSL/T1rho).

7. The method according to claim 1, wherein a spin-lock pulse is used together with a 2D or 3D MRI pulse sequence, for generating a T1rho image.

8. The method according to claim 7, wherein the spin-lock pulse is a rotary echo or other spin-lock pulse.

9. The method according to claim 7, wherein the 2D or 3D MRI pulse sequence is selected from the group consisting of spin echo (SE), fast spin echo (FSE), gradient echo (GRE), echo planar imaging (EPI) sequences and a 3D balanced fast field echo (bFFE) sequence.

10. The method according to claim 8, wherein rotary echo spin-lock pulse is used together with a 3D balanced fast field echo (bFFE) sequence for generating the T1rho weighted images.

11. The method according to claim 1, wherein the fibrosis is selected from the group consisting of liver, kidney, and other viscera fibrosis, preferably the fibrosis is liver fibrosis.

12. The method according to claim 11, wherein the fibrosis is liver fibrosis.

13. The method according to claim 1, wherein the subject is a human or an animal.

14. The method according to claim 1, wherein the normal standard of T1rho value is a T1rho value determined from a normal subject or a group of the normal subjects not having the fibrotic tissue or organ.

15. The method according to claim 1, wherein the current and other time points for acquiring T1rho weighted images can have a time interval as desired in the detection, such as four weeks, two months, three months, four months, five months, six months, one year, two years, etc.

16. The method according to claim 1, wherein the magnetic field strength used for acquiring a T1rho weighted image is selected from the group consisting of: low field below 1.5T, high field between 1.5T to 3T, and ultra high field over 3T.

17. A device for detecting or monitoring fibrosis in a tissue or an organ of a subject using nuclear magnetic resonance imaging, comprising:
a means for generating a spin-lattice relaxation time in the rotating frame (T1rho) weighted image of the region of interest (ROI) in said tissue or organ of the subject;
a means for measuring the T1rho value of the region of interest (ROI) in said tissue or organ of the subject by running an algorithm; and
a means for comparing the T1rho value in the tissue or organ of the subject with a normal standard of T1rho value in said tissue or organ, or with another T1rho value obtained from the tissue or organ of the subject at other time points;
wherein an increase of the T1rho value in the tissue or organ of the subject compared with the normal standard indicates that the subject is suffering from the tissue or organ fibrosis; or an increase or decrease of the T1rho value in the tissue or organ of the subject compared with the T1rho value obtained at other time points indicates the progression or regression of the tissue or organ fibrosis.

18. The device according to claim 17, wherein an extent of the increase of T1rho value from normal ranges indicates the severity of the fibrosis, and the rate of the increase or decrease of T1rho value over time indicates the rate of the progression or regression of the tissue or organ fibrosis.

19. The device according to claim 17, wherein the T1rho value is reflected by signal intensity on the T1rho weighted image.

20. The device according to claim 17, wherein the algorithm is selected from the group consisting of:
generating a T1rho map of the region of interest (ROI) on a pixel-by-pixel basis from the acquired T1rho-weighted images using a T1rho relaxation theoretical model, and obtaining the T1rho value of the region of interest (ROI) from the T1rho map; and
obtaining the T1rho value from all the pixels of the region of interest (ROI) in the acquired T1rho images using a T1rho relaxation theoretical model.
